(19) United States
(12) Patent Application Publication
(10) Pub. No.: US 2007/0149958 A1
(43) Pub. Date: Jun. 28, 2007

(54) Cryoprobe with exhaust heater

(75) Inventors: Russell L. DeLonzor, Pleasanton, CA (US); James B. Ross, Pleasanton, CA (US); Mathew J. Nalipinski, Pleasanton, CA (US); Keith Turner, Cambridge (GB); David J. Foster, Cambridge (GB); Tom A. Oakley, Cambridge (GB); Michael R. Cane, Cambridge (GB)

Correspondence Address: Crockett & Crockett;Suite 400, 24012 Calle De La Plata, Laguna Hills, CA, 92653, US

(73) Assignee: Sanarus Medical, Inc.

(21) Appl. No.: 11/317,970

(22) Filed: Dec. 23, 2005

(51) Int. Cl. A61B 18/02

(52) U.S. Cl. 606/21; 606/23

(57) ABSTRACT

A cryoprobe having an exhaust heater placed in fluid communication with exhausting cryogen to heat the cryogen prior to being vented to the atmosphere.

### FIELD OF THE INVENTION

The inventions described below relate to the field of cryoprobes and more specifically to cryoprobes used in surgical procedures.

### BACKGROUND OF THE INVENTION

Cryosurgical probes are used to treat a variety of diseases. The cryosurgical probes quickly freeze diseased body tissue masses, causing the tissue to die after which it will be absorbed by the body, expelled by the body or sloughed off. Cryothermal treatment is currently used to treat prostate cancer and benign prostate disease, breast tumors and breast cancer, liver tumors and cancer, glaucoma and other eye diseases. Cryosurgery is also proposed for the treatment of a number of other diseases.

A variety of cryosurgical instruments, referred to as cryoprobes, cryosurgical ablation devices, and cryostats and cryocoolers, have been available for cryosurgery. The preferred device uses Joule-Thomson cooling in devices known as Joule-Thomson cryostats. These devices take advantage of the fact that most gases, when rapidly expanded, become extremely cold. In these devices, a high pressure gas such as gaseous argon or gaseous nitrogen is expanded through a nozzle inside a small cylindrical sheath made of steel, and the Joule-Thomson expansion cools the steel sheath to sub-freezing cryogenic temperature very rapidly. Instead of gas, other cryosurgical instruments use liquid cryogen to quickly freeze diseased body tissue.

In cryosurgical instruments, it is important to prevent exhaust fluid from transferring heat to the inlet fluid as much as practical. Some cryosurgical instruments have exhaust cryogen fluid lines running in close proximity to inlet fluid lines. Current solutions to this heat transfer problem in cryosurgical instruments have been to flow the exhaust fluid in a separate tube or con-annularly with the inlet fluid for the length of the outer rigid tube to the handle in a cryprobe and then have the exhaust fluid flow back to the cryoprobe console in a separate line to be vented to the atmosphere. This solution, however, results in the cryoprobe having two bulky super-insulated lines, one for inlet fluid and the other for exhaust fluid. What is needed is a cryosurgical instrument system and method of use that prevents exhaust fluid from transferring heat to the inlet fluid as much as practical while also allowing exhaust fluid to be vented to the atmosphere without a second bulky exhaust line.

### SUMMARY

The cryoprobes and method their use described below prevent exhaust fluid from transferring heat to the inlet fluid as much as practical while also allowing exhaust fluid to be vented to the atmosphere. in an efficient manner. The cryoprobes include a probe with structures that permit the surgeon to secure and form an ice mass of a suspect mass or tumor. The probe is provided with a rigid outer tube, an inlet tube and a sharp distal segment. To secure the tumor to the probe, the surgeon pierces the tumor with the distal segment. Inlet tubing extending within the rigid outer tube directs coolant to the distal tip to cool the tip. A handle comprising an exhaust heater for heating exhaust fluid is disposed about the proximal end of the rigid outer tube. The exhaust heater is placed in fluid communication with exhausting cryogen is heated prior to being vented to atmosphere.

### BRIEF DESCRIPTION OF THE DRAWINGS

FIG. 1 illustrates a cryosurgical procedure for treating benign tumors in the breast.

FIG. 2 illustrates a detailed sectional view of a cryoprobe with an exhaust fluid heater.

### DETAILED DESCRIPTION OF THE INVENTION

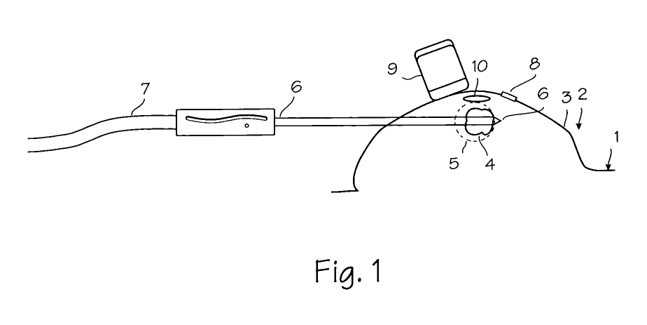

FIG. 1 illustrates a cryosurgical procedure for treating benign tumors in the breast. The patient 1 and the patient's breast 2 and skin 3 of the breast are shown schematically. The fibroadenoma 4 is located within the breast, surrounded by soft tissue and fatty tissue. The fibroadenoma is a well defined, hard mass ranging in size from 3 to 40 mm in diameter. The purpose of the procedure is to form an ice mass 5 (the frozen mass of breast tissue) around the fibroadenoma, after which the natural healing processes of the body will result in resorption of the fibroadenoma by the patient's body. The ice mass is formed with a cryoprobe 6, which, as illustrated, is inserted through the skin and intervening breast tissue into the fibroadenoma, so that the distal tip extends through the fibroadenoma. A cryogen supply hose 7 is attached to the cryoprobe and serves to supply cryogen from a cryogen source to the cryoprobe. The cryogen may be gaseous or liquid depending on the type of cryoprobe. The gas or liquid used for cooling may include argon, nitrogen, carbon dioxide, air, nitrous oxide, freon, chlorofluorocarbons (CFC's), perflourocarbons or any other suitable coolant. Gas may be provided through a cryosurgical system such as Endocare's Cryocare® cryosurgical systems. The cryoprobe may include a temperature sensor, which directly or indirectly measures the temperature of the cryoprobe. A temperature sensor 8 may be used during the surgery to monitor skin temperature, so that surgeons can avoid causing frost-bite on the patient's skin. An ultrasound probe 9 is used during the procedure to visualize the formation, growth, and melting of the iceball that is formed within the breast when the cryoprobe is energized. The iceball is highly echogenic, so that its formation is very clearly visualized. The image of the iceball is displayed on a display screen provided with the ultrasound probe. An insulating mass 10 of saline or other inert substance may be injected into the breast, between the fibroadenoma and the skin to protect the skin from freezing when the fibroadenoma is frozen.

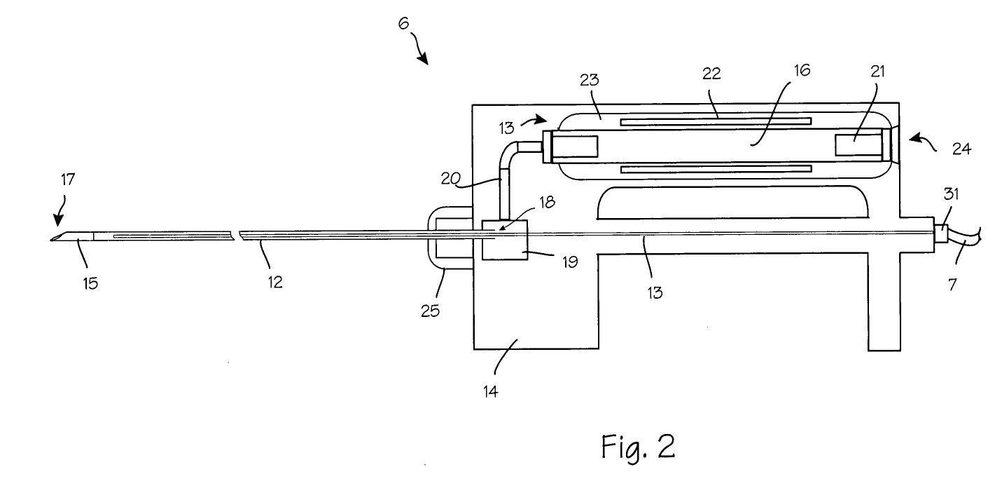

FIG. 2 illustrates a detailed sectional view of a cryoprobe with an exhaust fluid heater 11. The cryoprobe comprises a rigid outer tube 12, an inner coolant inlet tube 13 and a suitable handle 14 mounted on the proximal end of the outer tube. A trocar mounting nut 25 couples the outer tube to the handle. A short rigid penetrating segment 15 extends distally from the distal end of the outer tube. The coolant inlet 13 tube passes through the outer tube, extending to the distal end of the outer tube, terminating just proximal of the distal tip of the penetrating segment leaving a chamber 16 between the distal end of the inlet tube 13 and the proximal end of the penetrating element. The inlet tube may terminate by merely a straight cut or have a small nozzle of smaller internal diameter than the immediately upstream portion of the inlet tube. The outer tube is made from stainless steel. However, the outer tube may also be manufactured from aluminum, brass, ceramics or MRI compatible materials. The inlet tube 13 is made from a polyetheretherketon (PEEK) or other lower thermally conductive material having a pressure and temperature capability sufficient for the pressures and temperatures anticipated for the particular application. Use of lower thermally conductive materials for the inlet tube 13 reduces the amount of cryogen required by the cryoprobe. Suitable inlet tube 13 material includes fluoropolymer tubing (FEP), Teflon®, polyimide and polyurethane.

The outer tube has an outer diameter of about 2.7 mm, an internal diameter of about 2.4 mm, and a length of about 40 mm. The inlet tube 13 has an outer diameter of about 0.76 mm and an inner diameter of about 0.64 mm. These dimensions may vary depending on the materials used and the application for the cryoprobe. The penetrating segment comprises a sharp distal tip 17. As can be seen from the sectional view, the sharp distal tip is solid and adapted for piercing through a tumor. The length of the penetrating segment is chosen to be approximately the same size as the target tissue mass to be treated. This penetrating segment is forced into a lesion or tumor. An annular cavity 18 or lumen is created by the outer surface of the inlet tube and the inner surface of the rigid outer tube.

The liquid exiting the orifice of the inlet tube 13 counterflows along the annular cavity to an exhaust manifold 19 in fluid communication with the outer tube. The exhaust manifold isolates the cryogen exhaust from the annular cavity and directs the exhaust fluid through an exhaust line 20 into an exhaust heating chamber. The exhaust heating chamber is bounded on its distal end and proximal end by plugs 21. A heating element 22 in electrical communication with a power source is placed in thermal communication with the heating chamber allowing the exhaust heater to warm exhausting cryogen. Insulation 23 is disposed between the heating element and the outer surface of the handle in order to prevent the surface of the handle from becoming hot. An exhaust port 24 or vent is disposed on the proximal end of the heating chamber in fluid communication with the heating chamber allowing heated cryogen to safely vent to the atmosphere from the chamber.

The heating element may comprise resistance wire such as nichrome, self-regulating resistive polymers or other electrical restive materials. A console is provided and placed in electrical communication with the power source and placed in fluid communication with a cryogen source. The console has a control system that is able to regulate the use of power, the temperature of the probe, the temperature of the exhaust heater and the flow of cryogen to the cryoprobe.

When the cryoprobe is in use, the inlet tube 13 is placed in fluid communication with a lightly pressurized cryogen source by means of an inlet fitting. The cryogen is supplied to the assembly through a pressure fitting 31, flows through a fluid supply line, flows through the inlet tube and exits the distal end of the inlet tube 13. The distal end of the inlet tube 13 is exposed to a cavity at the distal end of the outer tube closed by the rigid penetrating segment. After expanding in the chamber, the fluid is at lower pressure and exhausts over the exhaust pathway which includes flow over outside of the inlet tube. The liquid nitrogen cools the distal tip of the probe to temperatures as low as −196° C. when steady flow has been established. The cryogen cools the inner surface of the rigid penetrating segment, thereby cooling the outer surface of the segment. The outer surface of the penetrating segment is placed against the targeted tissue to be cooled by the physician and the targeted tissue becomes an ice mass. Fluid flowing past the outer surface of the inlet tube is placed in contact with the helical-shaped baffle creating a turbulent helical flow path and forcing the cryogen torwards the inner surface of the outer tube. Turbulent fluid flow provides for improved heat transfer between the cryoprobe and targeted tissue. As the liquid nitrogen boils, the exhaust gas flows through the remainder of the exhaust gas pathway. Depending on the flow rates of the nitrogen, boiling can occur once the nitrogen flows past the baffle. In order to minimize cryogen consumption, flow rates can be reduced to a level where the nitrogen is about 90% vapor by the time it reaches the handle.

Once the exhaust fluid enters the handle of the cryoprobe, it is diverted away from the inlet tube to the heating chamber by the manifold. When the cryogen reaches the heating chamber, the exhaust heater heats the cryogen to a safe temperature that does not cause injury to a user of the cryoprobe when the cryogen is vented out of the handle. Safe temperatures may include temperatures ranging between about 32° F. to about 110° F. For example, a 150 watt heating element would be needed to heat the exhausting cryogen to a temperature of about 50° F. Once the cryogen is heated to a safe temperature, the cryogen is then vented to the atmosphere through the exhaust port.

While the preferred embodiments of the devices and methods have been described in reference to the environment in which they were developed, they are merely illustrative of the principles of the inventions. other embodiments and configurations may be devised without departing from the spirit of the inventions and the scope of the appended claims.

We claim:

1. A cryoprobe for use in a cryosurgical procedure comprising:
a rigid outer tube;
a coolant inlet tube disposed within the outer tube;
a short rigid penetrating segment extending distally from a distal end of the outer tube;
a handle disposed about a proximal end of the outer tube; and
an exhaust heater disposed within the handle, said exhaust heater placed in thermal communication with exhausting cryogen and adapted to warm exhausting cryogen.

2. The cryoprobe of claim 1 wherein the exhaust heater comprises a heating chamber placed in fluid communication with the rigid tube and a heating element placed in thermal communication with the heating chamber.

3. The cryoprobe of claim 2 further comprising a manifold disposed in the handle in fluid communication with the outer tube, said manifold adapted to direct exhaust cryogen to the heating chamber.

4. The cryoprobe of claim 1 wherein the exhaust heater is adapted to heat the exhaust cryogen to a temperature in the range of about 32° C. to about 110° F.

5. The cryoprobe of claim 2 wherein the heating element comprises resistance wire.

6. A system comprising:
a cryogen source;
a cryogen supply line in fluid communication with the cryogen source; and
a cryoprobe in fluid communication with the supply line, said cryoprobe a having a rigid outer tube;
a coolant inlet tube in fluid communication with the cryogen source disposed within the outer tube;
a short rigid penetrating segment extending distally from a distal end of the outer tube;
a handle disposed about a proximal end of the outer tube; and
an exhaust heater disposed within the handle, said exhaust heater placed in thermal communication with exhausting cryogen and adapted to warm exhausting cryogen.

7. The system of claim 6 wherein the exhaust heater comprises a heating chamber placed in fluid communication with the rigid tube and a heating element placed in thermal communication with the heating chamber.

8. The system of claim 6 wherein the cryoprobe further comprises a manifold disposed in the handle in fluid communication with the outer tube, said manifold adapted to direct exhaust cryogen to the heating chamber.

9. The system of claim 1 wherein the exhaust heater is adapted to heat the exhaust cryogen to a temperature in the range of about 32° C. to about 110° F.

10. The system of claim 7 wherein the heating element comprises resistance wire.

11. The system of claim 1 wherein the cryogen source comprises a cryogen selected from the group consisting of argon, nitrogen, carbon dioxide, air, freon, nitrous oxide, chlorofluorocarbons and perflourocarbons.

12. A method of performing a cryosurgical procedure comprising:
providing a cryoprobe having an outer rigid tube, an inlet tube disposed within the outer tube, a handle disposed about a proximal end of the outer tube and an exhaust heater disposed within the handle;
performing a cryosurgical procedure using the cryprobe;
heating exhausting cryogen in the handle with the exhaust heater prior to venting the cryogen to the atmosphere; and
venting the cryogen to the atmosphere through an exhaust port disposed in the handle.

13. The method of claim 12 wherein the step of heating exhausting cryogen further comprises heating the cryogent to a safe temperature.
